(19) United States
(12) Patent Application Publication
(10) Pub. No.: US 2024/0420291 A1
(43) Pub. Date: Dec. 19, 2024

(54) IMAGE PROCESSING APPARATUS, OPERATION METHOD OF IMAGE PROCESSING APPARATUS, PROGRAM, AND MEDICAL IMAGE PROCESSING SYSTEM

(71) Applicant: FUJIFILM Healthcare Corporation, Chiba (JP)

(72) Inventors: Atsuro SUZUKI, Chiba (JP); Tomoki AMEMIYA, Chiba (JP); Yukio KANEKO, Chiba (JP); Suguru YOKOSAWA, Chiba (JP); Toru SHIRAI, Chiba (JP)

(73) Assignee: FUJIFILM Healthcare Corporation, Chiba (JP)

(21) Appl. No.: 18/736,475

(22) Filed: Jun. 6, 2024

(30) Foreign Application Priority Data
Jun. 14, 2023 (JP) 2023-098130

(51) Int. Cl. G06T 5/70; A61B 5/00; A61B 5/055; G06T 7/00

(52) U.S. Cl. CPC G06T 5/70; A61B 5/055; A61B 5/7203; A61B 5/7267; G06T 7/0016; G06T 2200/24; G06T 2207/10088; G06T 2207/20092; G06T 2207/30004; G06T 2207/30168

(57) ABSTRACT

Provided are an image processing apparatus, an operation method of an image processing apparatus, a program, and a medical image processing system, in which denoising processing corresponding to a preference of a user is realized.
An image processing apparatus acquires user identification information, acquires a first imaging condition, acquires a first image to which the first imaging condition is applied, executes denoising processing on the first image by applying a first denoising strength, to generate a first denoised image, acquires, from among second signal-to-noise ratios corresponding to a plurality of second imaging conditions, a second signal-to-noise ratio corresponding to a second imaging condition that is identical to or close to the first imaging condition, as a first signal-to-noise ratio, and sets a denoising strength based on the first signal-to-noise ratio, as the first denoising strength.

### CROSS-REFERENCE TO RELATED APPLICATION

The present application claims priority under 35 U.S.C § 119(a) to Japanese Patent Application No. 2023-098130 filed on Jun. 14, 2023, which is hereby expressly incorporated by reference, in its entirety, into the present application.

### BACKGROUND OF THE INVENTION

### 1. Field of the Invention

The present disclosure relates to an image processing apparatus, an operation method of an image processing apparatus, a program, and a medical image processing system.

### 2. Description of the Related Art

JP2022-6869A describes a medical image apparatus comprising an MRI apparatus as an imaging apparatus. In the medical image apparatus described in JP2022-6869A, in a case in which the magnitude of the noise is different for each region of the image, a denoising filter having a relatively large strength is applied to a region having a relatively large noise level, and a denoising filter having a relatively small strength is applied to a region having a relatively small noise level. It should be noted that MRI is an abbreviation for Magnetic Resonance Imaging, which is an English notation.

In addition, in the medical image apparatus described in JP2022-6869A, a relationship between a region selection pattern selected by the user and an imaging condition is stored. As a result, in a case in which the imaging condition that is the same as the imaging condition applied in the past is applied, it is possible to select the region selection pattern selected in the past.

Further, the medical image apparatus described in JP2022-6869A comprises a slide bar to which the region selection pattern is associated with each slide position. The user can stop the slide position while sliding the slide bar and viewing an image having a high image quality displayed on a display device, and can select the region selection pattern in which a desired image having a high image quality is obtained.

### SUMMARY OF THE INVENTION

However, in the image processing apparatus in the related art, the denoising strength is set before the imaging is performed, and the denoising processing is applied to the image generated after the imaging ends. In a case in which the user confirms the image to which the denoising is applied and wants to change the denoising strength, the denoising strength is set again, and re-imaging is performed. In an actual clinical site, it is not realistic to perform the re-imaging.

In addition, the denoising strength recommended by a manufacturer of the image processing apparatus does not always match a preference of the user, such as a doctor or a technician, and it is desired to optimize the denoising strength corresponding to the preference of the user.

The present disclosure has been made in view of such circumstances, and an object of the present disclosure is to provide an image processing apparatus, an operation method of an image processing apparatus, a program, and a medical image processing system, in which denoising processing in which a denoising strength corresponding to a preference of a user is applied is realized.

A first aspect of the present disclosure relates to an image processing apparatus comprising: a processor configured to: acquire user identification information; acquire a first imaging condition applied to an imaging apparatus; acquire a first image generated by being captured using the imaging apparatus to which the first imaging condition is applied; set a first denoising strength applied to denoising processing on the first image; and execute the denoising processing on the first image by applying the first denoising strength, to generate a first denoised image, in which the processor is configured to: acquire, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition, as a first signal-to-noise ratio; and set a denoising strength derived based on the first signal-to-noise ratio, as the first denoising strength.

With the image processing apparatus according to the first aspect, the second signal-to-noise ratio corresponding to the second imaging condition that is identical to the first imaging condition or the second imaging condition close to the first imaging condition is acquired as the first signal-to-noise ratio from among the plurality of second signal-to-noise ratios derived from the plurality of second images generated in the plurality of times of past imaging for each user, the plurality of second signal-to-noise ratios being associated with the second imaging conditions in the past imaging. As a result, the denoising processing in which the first denoising strength corresponding to the first signal-to-noise ratio preferred by the user is applied is realized.

A second aspect relates to the image processing apparatus according to the first aspect, in which the processor may be configured to acquire the first signal-to-noise ratio corresponding to the second imaging condition that is identical to the first imaging condition or the second imaging condition that is close to the first imaging condition, with reference to a signal-to-noise ratio database in which the plurality of second imaging conditions are stored in association with the plurality of second signal-to-noise ratios.

A third aspect relates to the image processing apparatus according to the second aspect, in which the processor may be configured to, in a case in which the first denoising strength is changed, update the signal-to-noise ratio database in accordance with a second denoising strength after the change.

A fourth aspect relates to the image processing apparatus according to any one of the first to third aspects, in which the processor may be configured to: execute a plurality of times of the denoising processing with different denoising strengths on the first image, to generate a plurality of third denoised images; derive a third signal-to-noise ratio from each of the plurality of third denoised images; and set a third denoising strength applied to the third denoised image in which the third signal-to-noise ratio having a smallest difference from the first signal-to-noise ratio is derived, as the first denoising strength.

A fifth aspect relates to the image processing apparatus according to any one of the first to third aspects, in which the processor may be configured to: calculate a fourth signal-to-noise ratio from the first image; and set a fourth denoising strength corresponding to the fourth signal-to-noise ratio, as the first denoising strength, by using a function representing a relationship between a signal-to-noise ratio and a denoising strength.

A sixth aspect relates to the image processing apparatus according to any one of the first to fifth aspects, in which the processor may be configured to: display, on a display device, the first denoised image; display, on the display device, options representing an evaluation of the first denoising strength applied to the denoising processing on the first denoised image; acquire a first evaluation signal representing the evaluation of the first denoising strength selected from among the options; and perform feedback of the evaluation of the first denoising strength represented by the first evaluation signal with respect to the setting of the first denoising strength.

A seventh aspect relates to the image processing apparatus according to any one of the first to sixth aspects, in which the processor may be configured to: display, on a display device, a fifth denoised image generated by executing the denoising processing by applying the first denoising strength to a past image generated by being captured in the past by applying a fifth imaging condition that is identical to the first imaging condition or a fifth imaging condition that is close to the first imaging condition; acquire a fifth evaluation signal representing an evaluation of a fifth denoising strength applied to the denoising processing on the fifth denoised image; and determine whether to maintain or change the first denoising strength in accordance with the evaluation of the fifth denoising strength represented by the fifth evaluation signal.

An eighth aspect relates to the image processing apparatus according to the seventh aspect, in which a normal image generated by imaging a normal imaging target in the past may be applied to the past image.

A ninth aspect relates to the image processing apparatus according to the seventh aspect, in which an identical-imaging target image generated by imaging an identical imaging target in the past by applying a sixth imaging condition that is identical to the first imaging condition or a sixth imaging condition that is close to the first imaging condition may be applied to the past image, and the processor may be configured to display, on the display device, a sixth denoised image obtained by executing the denoising processing on the identical-imaging target image, and a sixth denoising strength applied to the denoising processing executed on the identical-imaging target image.

A tenth aspect provides the image processing apparatus according to the ninth aspect, in which the processor may be configured to: acquire a sixth evaluation signal representing an evaluation of the sixth denoising strength; and determine whether to maintain or change the first denoising strength in accordance with the evaluation of the sixth denoising strength represented by the sixth evaluation signal.

An eleventh aspect of the present disclosure relates to an operation method of an image processing apparatus to which a computer provided with a processor is applied, the operation method comprising: via the processor, acquiring user identification information; acquiring a first imaging condition applied to an imaging apparatus; acquiring a first image generated by being captured using the imaging apparatus to which the first imaging condition is applied; setting a first denoising strength applied to denoising processing on the first image; and executing the denoising processing on the first image by applying the first denoising strength, to generate a first denoised image, in which, in a case of setting the first denoising strength, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition is acquired as a first signal-to-noise ratio, and a denoising strength derived based on the first signal-to-noise ratio is set as the first denoising strength.

A twelfth aspect of the present disclosure relates to a program causing a computer to realize: a function of acquiring user identification information; a function of acquiring a first imaging condition applied to an imaging apparatus; a function of acquiring a first image generated by being captured using the imaging apparatus to which the first imaging condition is applied; a function of setting a first denoising strength applied to denoising processing on the first image; and a function of executing the denoising processing on the first image by applying the first denoising strength, to generate a first denoised image, in which, in the function of setting the first denoising strength, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition is acquired as a first signal-to-noise ratio, and a denoising strength derived based on the first signal-to-noise ratio is set as the first denoising strength.

A non-transitory computer-readable recording medium that is a tangible object storing the program according to the twelfth aspect is also included in the present disclosure.

A thirteenth aspect of the present disclosure relates to a medical image processing system comprising: an image processing apparatus configured to execute defined image processing on a medical image generated by imaging a subject using an imaging apparatus, in which the image processing apparatus includes a processor configured to: acquire user identification information; acquire a first imaging condition applied to an imaging apparatus; acquire a first image generated by being captured using the imaging apparatus to which the first imaging condition is applied; set a first denoising strength applied to denoising processing on the first image; and execute the denoising processing on the first image by applying the first denoising strength, to generate a first denoised image, and the processor is configured to: acquire, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition, as a first signal-to-noise ratio; and set a denoising strength derived based on the first signal-to-noise ratio, as the first denoising strength.

According to the present disclosure, the second signal-to-noise ratio corresponding to the second imaging condition that is identical to the first imaging condition or the second imaging condition close to the first imaging condition is acquired as the first signal-to-noise ratio from among the plurality of second signal-to-noise ratios derived from the plurality of second images generated in the plurality of times of past imaging for each user, the plurality of second signal-to-noise ratios being associated with the second imaging conditions in the past imaging. As a result, the denoising processing in which the first denoising strength corresponding to the first signal-to-noise ratio preferred by the user is applied is realized.

### BRIEF DESCRIPTION OF THE DRAWINGS

FIG. 1 is an overall configuration diagram of an MRI apparatus according to an embodiment.

FIG. 2 is a functional block diagram illustrating an electric configuration of a computer illustrated in FIG. 1.

FIG. 3 is a block diagram illustrating a hardware configuration of the electric configuration of the computer illustrated in FIG. 2.

FIG. 4 is a flowchart illustrating a procedure of construction of an SN ratio database according to a first example.

FIG. 5 is a flowchart illustrating a procedure of construction of an SN ratio database according to a second example.

FIG. 6 is a flowchart illustrating a procedure of an image processing method according to the embodiment.

FIG. 7 is a functional block diagram illustrating a first configuration example of a denoising strength acquisition unit illustrated in FIG. 2.

FIG. 8 is an explanatory diagram of a background of a region of interest.

FIG. 9 is an explanatory diagram of a pixel value of background noise.

FIG. 10 is a functional block diagram illustrating a second configuration example of the denoising strength acquisition unit illustrated in FIG. 2.

FIG. 11 is a table illustrating a result of calculating an SN ratio of a denoised image generated by applying a plurality of denoising strengths to a numerical phantom having different SN ratios.

FIG. 12 is an explanatory diagram of a function representing a denoising strength.

FIG. 13 is a functional block diagram illustrating an electric configuration of a computer according to a first modification example.

FIG. 14 is an explanatory diagram illustrating an example of a user interface applied to feedback of the denoising strength.

FIG. 15 is an explanatory diagram illustrating a first example of a confirmation screen before automatic adjustment of the denoising strength.

FIG. 16 is an explanatory diagram illustrating a second example of the confirmation screen before the automatic adjustment of the denoising strength.

### DESCRIPTION OF THE PREFERRED EMBODIMENTS

Hereinafter, a preferred embodiment of the present invention will be described in detail with reference to the accompanying drawings. It should be noted that, in the following description and the accompanying drawings, the identical reference numerals denote the identical components, and the description thereof will be omitted. In addition, in the following embodiment, in a case in which a plurality of components are described and listed, it can be interpreted that at least one of the plurality of components is included.

### First Embodiment

### Configuration Example of MRI Apparatus

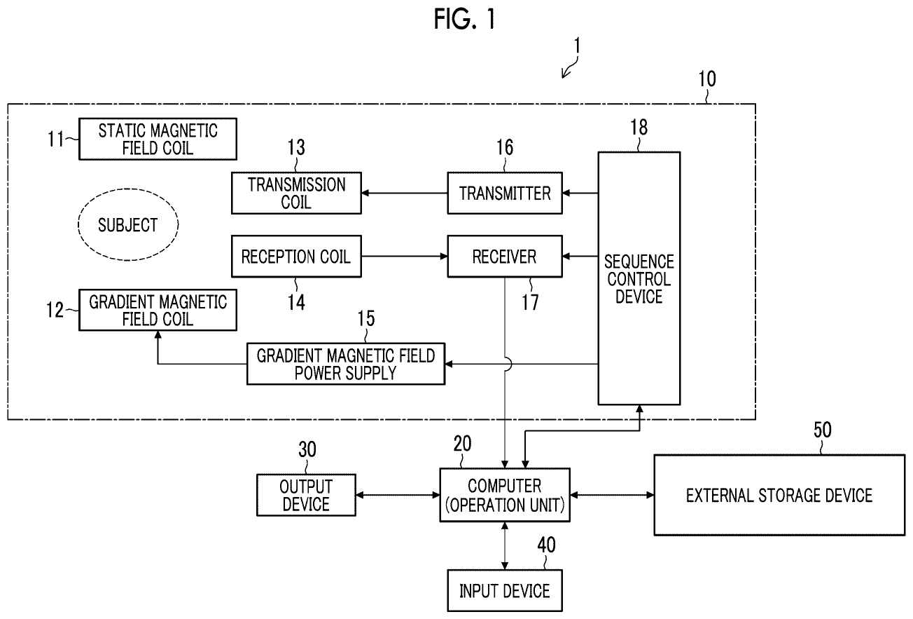

FIG. 1 is an overall configuration diagram of an MRI apparatus according to the embodiment. Hereinafter, the MRI apparatus will be described as one embodiment of the present disclosure, but the technology of the present disclosure can also be applied to modalities such as an X-ray CT apparatus and a positron emission tomography apparatus. It should be noted that the CT is an abbreviation for Computed Tomography. The positron emission tomography may be referred to as PET using an abbreviation for Positron Emission Tomography, which is an English notation.

An MRI apparatus 1 illustrated in FIG. 1 comprises a measurement device 10, a computer 20, an output device 30, an input device 40, and an external storage device 50. The measurement device 10 comprises a static magnetic field coil 11, a gradient magnetic field coil 12, and a gradient magnetic field power supply 15. The static magnetic field coil 11 generates a static magnetic field in a space in which a subject is placed. The gradient magnetic field coil 12 provides a magnetic field gradient with respect to the static magnetic field generated by the static magnetic field coil 11. The gradient magnetic field power supply 15 is a driving power supply of the gradient magnetic field coil 12.

The measurement device 10 comprises a transmission coil 13, a reception coil 14, a transmitter 16, and a receiver 17. The transmission coil 13 generates a high-frequency magnetic field in a measurement region of the subject. The transmitter 16 supplies a pulse current, which is an excited current, to the transmission coil 13. The reception coil 14 receives a nuclear magnetic resonance signal generated from the subject. The receiver 17 transmits the nuclear magnetic resonance signal received by the reception coil 14 to the computer 20. It should be noted that the nuclear magnetic resonance signal may be referred to as an echo signal.

There are MRI apparatuses 1 of a vertical magnetic field type and a horizontal magnetic field type depending on a direction of the generated static magnetic field. The static magnetic field coil 11 is adopted in various forms in accordance with the magnetic field type. The gradient magnetic field coil 12 comprises a plurality of coils that generate gradient magnetic fields respectively for three axial directions orthogonal to each other. The plurality of coils provided in the gradient magnetic field coil 12 are each driven by the gradient magnetic field power supply 15. The position information is added to the nuclear magnetic resonance signal generated from the subject due to the application of the gradient magnetic field.

It should be noted that, although FIG. 1 illustrates an aspect in which the transmission coil 13 and the reception coil 14 are individually provided, there may be an aspect in which one coil having a function of the transmission coil 13 and a function of the reception coil 14 is provided.

The measurement device 10 comprises a sequence control device 18. The sequence control device 18 controls the operations of the gradient magnetic field power supply 15 and the transmitter 16 to control generation timings of the gradient magnetic field and the high-frequency magnetic field. The sequence control device 18 controls the operation of the receiver 17 to control a reception timing of the nuclear magnetic resonance signal and to execute the measurement. A time chart of the control applied to the sequence control device 18 is referred to as an imaging sequence, is set in advance in accordance with the measurement, and is stored in a storage device or the like provided in the computer 20.

A computer comprising a processor, a memory, a storage device, and the like, such as a CPU, is applied to the computer 20. The computer 20 functions as a control device that controls the operations of the respective units of the measurement device 10 via the sequence control device 18. In addition, the computer 20 executes operation processing on the nuclear magnetic resonance signal received via the receiver 17 and the sequence control device 18, and functions as an operation device that acquires an image signal of a predetermined imaging region. The computer 20 may be a device constituting the MRI apparatus 1, or may be an external device being independent of the MRI apparatus 1.

The output device 30, the input device 40, and the external storage device 50 are electrically connected to the computer 20 such that the data communication can be performed. A display that displays a result of the operation processing executed by the computer 20 may be applied to the output device 30.

A liquid crystal display, an organic EL display, a projector, or the like may be applied to the output device 30. The output device 30 may be appropriately combined with the liquid crystal display, the organic EL display, the projector, or the like. It should be noted that the organic EL may be referred to as OEL using an abbreviation for Organic Electro-Luminescence.

The input device 40 is an interface through which an operator inputs conditions, parameters, or the like applied to the measurement, the operation processing, and the like. Examples of the input device 40 include a keyboard and a mouse. The display device and the input device that function as the output device 30 may be integrally configured by using a touch panel type display. The operator can input the parameters, such as the number of measured echoes, the echo time, and the echo interval, by using the input device 40.

The external storage device 50 stores data used in various types of the operation processing executed by the computer 20, data derived as a result of the operation processing, conditions applied to the operation processing, parameters applied to the operation processing, a program for executing the operation processing, and the like. The external storage device 50 may realize a part of a function of an internal storage device of the computer 20, or the internal storage device of the computer 20 may realize a part of the function of the external storage device 50.

In the nuclear magnetic resonance signal acquired by using the measurement device 10, the noise caused characteristics and imaging conditions of the MRI apparatus 1 is superimposed on the signal from the subject. The noise superimposed on the nuclear magnetic resonance signal deteriorates an image quality of a captured image of the subject generated from the nuclear magnetic resonance signal. The computer 20 has a function of executing noise reduction processing, which is processing of reducing the noise superimposed on the nuclear magnetic resonance signal, as signal processing on the nuclear magnetic resonance signal. The noise reduction processing may be referred to as denoising processing.

A trained learning model such as a CNN is applied to the denoising processing. It should be noted that CNN is an abbreviation for a Convolution Neural Network. The external storage device 50 stores an SN ratio database used in a case in which a filter strength applied to the denoising processing is derived. The filter strength may be referred to as a denoising strength.

The SN ratio database stores an SN ratio preferred by a user, such as a doctor or a technician, in the image in association with the imaging conditions applied in a case of imaging the subject. In the SN ratio database, the preferred SN ratio in the image can be read out with the imaging conditions as an index.

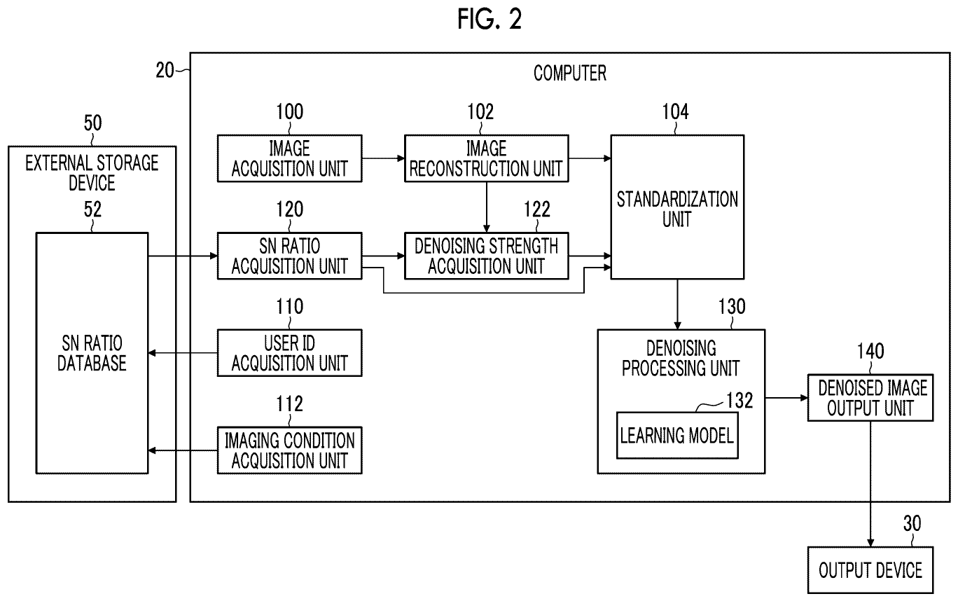

It should be noted that the SN ratio may be referred to as a signal-to-noise ratio. In FIG. 1, the SN ratio database is not illustrated. The SN ratio database is denoted by reference numeral 52 and illustrated in FIG. 2. It should be noted that the measurement device 10 illustrated in FIG. 1 is an example of an imaging apparatus, and the computer 20 is an example of an image processing apparatus and is an example of a component of a medical image processing system. Configuration example of computer

FIG. 2 is a functional block diagram illustrating an electric configuration of the computer illustrated in FIG. 1. The computer 20 comprises an image acquisition unit 100. The image acquisition unit 100 acquires an image represented by the nuclear magnetic resonance signal transmitted from the receiver 17 illustrated in FIG. 1. It should be noted that the image acquired by using the image acquisition unit 100 described in the embodiment illustrated in FIG. 2 is an example of a first image.

The computer 20 comprises an image reconstruction unit 102. The image reconstruction unit 102 generates a reconstructed image based on the image acquired via the image acquisition unit 100. Examples of the reconstructed image generated based on the image include a two-dimensional tomographic image. Here, the term “image” also means image data representing an image. The term “data” may also mean a signal.

The computer 20 comprises a standardization unit 104. The standardization unit 104 executes standardization processing on a pixel value of each pixel constituting the reconstructed image by applying a defined standardization constant. Details of the standardization processing will be described later.

The computer 20 comprises a user ID acquisition unit 110. The user ID acquisition unit 110 acquires a user ID used for identifying the user who executes the denoising processing on the standardized reconstructed image. Information for identifying the user, such as a name of the user and an identification number for each user, is applied to the user ID. The user ID acquisition unit 110 may adopt an aspect in which the user ID represented by a barcode, a two-dimensional code, or the like is read by using a reading device. It should be noted that the user ID described in the embodiment is an example of user identification information. ID is an abbreviation for Identification.

The computer 20 comprises an imaging condition acquisition unit 112. The imaging condition acquisition unit 112 acquires the imaging conditions applied to the imaging of the subject. The imaging represents a function of the measurement device 10 in which the subject is irradiated with the electromagnetic waves from the static magnetic field coil 11 or the like and the nuclear magnetic resonance signal is acquired by using the receiver 17. Examples of the imaging conditions include the number of imaging matrices and a sequence. It should be noted that the imaging condition acquired by using the imaging condition acquisition unit 112 described in the embodiment is an example of a first imaging condition.

The computer 20 comprises an SN ratio acquisition unit 120. The SN ratio acquisition unit 120 reads out the SN ratio corresponding to an imaging target part, the user ID, and the imaging condition from the SN ratio database 52 in which the SN ratio is stored in association with the imaging condition for each user. The SN ratio read out from the SN ratio database 52 is used in the operation processing as the preferred SN ratio for each user corresponding to the imaging condition. It should be noted that the imaging target part may be interpreted as an anatomical structure of the imaging target.

That is, the SN ratio database 52 stores the SN ratio measured with respect to an MR image generated in the plurality of times of past imaging, in association with the imaging part and the imaging condition accompanying the MR image for each user. A DICOM format can be applied to the imaging part and the imaging condition. It should be noted that the MR image which is captured and generated in the past and described in the embodiment, is an example of a second image, and the imaging condition of the past image is an example of a second imaging condition.

The SN ratio acquisition unit 120 searches for the SN ratio database 52 with the imaging target part, the user ID, and the imaging condition as indexes, to read out the preferred SN ratio for each user in accordance with these indexes. It should be noted that the SN ratio database 52 described in the embodiment is an example of a signal-to-noise ratio database. A plurality of SN ratios stored in the SN ratio database 52 described in the embodiment are examples of a plurality of second signal-to-noise ratios, and an SN ratio SN0 read out from the SN ratio database 52 is an example of a first signal-to-noise ratio acquired from among the plurality of second signal-to-noise ratios.

The computer 20 comprises a denoising strength acquisition unit 122. The denoising strength acquisition unit 122 acquires the denoising strength applied to the denoising processing, based on the SN ratio SN0 read out from the SN ratio database 52. Details of the acquisition of the denoising strength will be described later. It should be noted that the denoising strength applied to the denoising processing described in the embodiment is an example of a first denoising strength.

The standardization unit 104 executes the standardization processing on the reconstructed image by using the SN ratio acquired from the SN ratio acquisition unit 120 and the denoising strength acquired from the denoising strength acquisition unit 122.

The computer 20 comprises a denoising processing unit 130. The denoising processing unit 130 executes the denoising processing on the standardized reconstructed image to generate a denoised image. The denoised image is generated by removing at least a part of the noise from the reconstructed image in which the noise is included.

The denoising processing unit 130 comprises a trained learning model 132. The CNN is applied to the learning model 132. The learning model 132 generated by being trained, by using training data in which an image including the noise is used as input data and an image from which the noise is removed is used as correct answer data, to output the image form which the noise is removed, in response to the input of the image including the noise. The generation of the learning model 132 may be executed by using a computer being independent of the computer 20, or may be executed by using a learning model generation unit provided in the computer 20. In a case in which the computer 20 is provided with the learning model generation unit, the computer 20 may retrain the learning model 132. It should be noted that the denoising processing applied to the denoising processing unit 130 illustrated in FIG. 2 is performed, and the denoised image to be generated is an example of a first denoised image.

The computer 20 comprises a denoised image output unit 140. The denoised image output unit 140 converts the denoised image generated by using the denoising processing unit 130 into a signal format applied to the output device 30, and outputs the converted denoised image to the output device 30. For example, in a case in which the output device 30 is a display, the denoised image output unit 140 converts the denoised image into a video signal, and outputs the converted video signal to the output device 30.

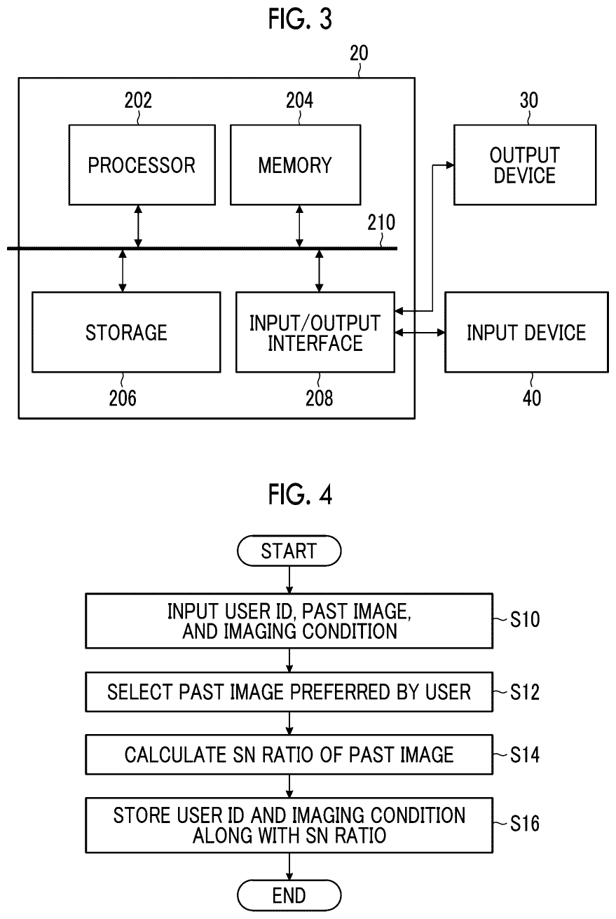

FIG. 3 is a block diagram illustrating a hardware configuration of the electric configuration of the computer illustrated in FIG. 2. The computer applied to the computer 20 may be a personal computer or a workstation. The computer may be a virtual machine. The computer 20 may be configured by using a plurality of computers.

The computer 20 comprises a processor 202, a memory 204 as a main storage device, a storage 206 as an auxiliary storage device, an input/output interface 208, and a bus 210. The processor 202 includes a CPU. The processor 202 may include a GPU. It should be noted that the CPU is an abbreviation for a Central Processing Unit. GPU is an abbreviation for a Graphics Processing Unit.

The processor 202 is connected to the memory 204, the storage 206, and the input/output interface 208 via the bus 210. The input device 40 and the output device 30 are connected to the computer 20 via the input/output interface 208.

The processor 202 realizes various functions by executing a program stored in the memory 204. The input/output interface 208 includes a communication interface connectable to a network, a connection interface connectable to an external device, and the like. Examples of the network include a local area network in a medical facility. As the connection interface connectable to the external device, for example, a universal serial bus and HDMI may be applied. It should be noted that the universal serial bus may be referred to as USB using an abbreviation for Universal Serial Bus. HDMI is an abbreviation for High-Definition Multimedia Interface. USB and HDMI are registered trademarks.

The processor 202 communicates with various external devices of the MRI apparatus 1 via the input/output interface 208, to transmit and receive necessary information. It should be noted that various external devices are not illustrated.

The output device 30 may include a display that displays various types of information, in addition to a medical image captured by using the MRI apparatus 1. The output device 30 is used as a part of a GUI in a case of receiving the input from the input device 40. It should be noted that the number of the output devices 30 is not limited to one, and a multi-display form comprising a plurality of displays can also be adopted. GUI is an abbreviation for Graphical User Interface.

### Construction of SN Ratio Database

FIG. 4 is a flowchart illustrating a procedure of construction of the SN ratio database according to a first example. The construction of the SN ratio database 52 illustrated in FIG. 2 is performed by using an information processing apparatus to which the computer is applied, and the following procedure is applied. The procedure of the construction of the SN ratio database according to the first example is applied in a case in which the past image is prepared for a plurality of the subjects. The past image referred herein means an MR image captured and generated in the past.

In first information input step S10, the past image is input to the information processing apparatus. In first information input step S10, the user ID and the imaging condition are input to the information processing apparatus for each of a plurality of past images.

In past image selection step S12, the user inputs selection information of the past image on which the denoising processing preferred by the user is performed, to the information processing apparatus. For example, in past image selection step S12, the past image on which the denoising processing preferred by one or more users is performed may be selected from among the past images subjected to the denoising processing. In past image selection step S12, the past image subjected to the denoising processing preferred by one or more users may be selected from among a plurality of past images generated by executing the denoising processing with different denoising strengths for any one of the past images.

In SN ratio calculation step S14, the SN ratio of the past image selected in past image selection step S12 is calculated. Details of the calculation of the SN ratio will be described later. A known method may be applied to the calculation of the SN ratio.

In SN ratio storage step S16, the imaging part and the imaging condition are stored for each user in association with the SN ratio calculated in SN ratio calculation step S14. As described above, the SN ratio database 52 is constructed and stored in the external storage device 50 illustrated in FIG. 1. The procedure of the construction of the SN ratio database 52 illustrated in FIG. 4 may be applied as a procedure of update of the SN ratio database 52.

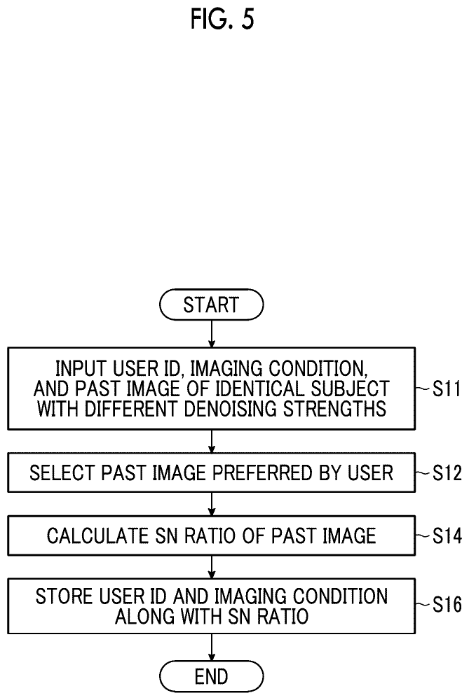

FIG. 5 is a flowchart illustrating a procedure of construction of the SN ratio database according to a second example. The procedure of the construction of the SN ratio database according to the second example is applied in a case in which a plurality of past images with different denoising strengths are prepared for the identical subject.

In the procedure of the construction of the SN ratio database illustrated in FIG. 5, second information input step S11 is executed instead of first information input step S10 illustrated in FIG. 4. In second information input step S11, the imaging part, the user ID, and the imaging condition are input for each of the plurality of past images of the identical subject.

After second information input step S11, past image selection step S12, SN ratio calculation step S14, and SN ratio storage step S16 are executed, and the SN ratio database 52 is constructed. The pieces of processing of past image selection step S12, SN ratio calculation step S14, and SN ratio storage step S16 are the same as in the procedure of the construction of the SN ratio database according to the second example illustrated in FIG. 4, and the description thereof will be omitted.

### Detailed Description of Denoising Processing

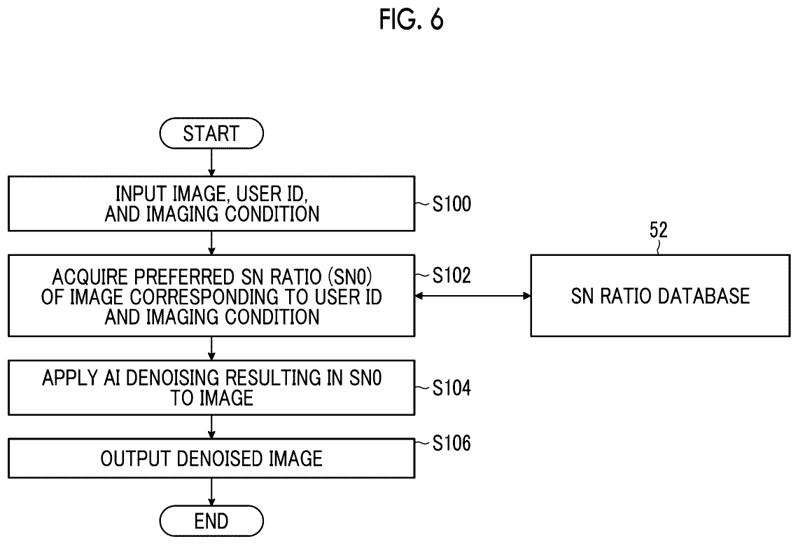

FIG. 6 is a flowchart illustrating a procedure of an image processing method according to the embodiment. In the image processing method applied to the MRI apparatus 1 according to the embodiment, the denoising strength applied to the denoising processing is automatically set with reference to the SN ratio of the MR image captured in the past by the identical user. It should be noted that the image processing method according to the embodiment is understood as an operation method of the image processing apparatus.

In third information input step S100, the image acquisition unit 100 illustrated in FIG. 2 acquires a processing target image. The information on the imaging part can be acquired in a case of acquiring the processing target image. In addition, in third information input step S100, the user ID acquisition unit 110 acquires the user ID of the user who performs the imaging to acquire the processing target image. Further, in third information input step S100, the imaging condition acquisition unit 112 acquires the imaging condition applied to the imaging for acquiring the processing target image.

In SN ratio acquisition step S102, the SN ratio acquisition unit 120 acquires the SN ratio SN0 preferred by the user from the SN ratio database 52 with reference to the SN ratio database 52 using the user ID and the imaging condition acquired in third information input step S100 as the indexes. The SN ratio SN0 preferred by the user is an example of a first signal-to-noise ratio.

In a case in which the SN ratio corresponding to the user ID and the imaging condition is not stored in the SN ratio database 52, the SN ratio of the past image having a close imaging condition may be acquired by the identical user. Examples of the close imaging condition include an imaging condition in which a part of a plurality of components match and an imaging condition in which a difference between the index values is within a defined range. That is, it can be understood that the close imaging condition has a difference to the extent that the same result as the identical imaging condition is obtained in the setting of the SN ratio and the setting of the denoising strength.

In denoising processing step S104, the standardization unit 104 executes the standardization processing on the image, which is a target of the denoising processing, by applying the SN ratio SN0 acquired in SN ratio acquisition step S102 and the denoising strength corresponding to the SN ratio SN0. In addition, in denoising processing step S104, the denoising processing unit 130 executes the denoising processing on the image subjected to the standardization processing, by using the learning model 132. AI denoising illustrated in FIG. 6 represents the denoising processing using the learning model 132. It should be noted that Al is an abbreviation for Artificial Intelligence.

In denoised image output step S106, the denoised image output unit 140 converts the denoised image subjected to the denoising processing into the output format of the output device 30, and outputs the converted denoised image to the output device 30. In a case in which the denoised image is output in denoised image output step S106, the procedure of the image processing method ends.

### Specific Example of Denoising Strength Adjustment

### Multi-Stage Type

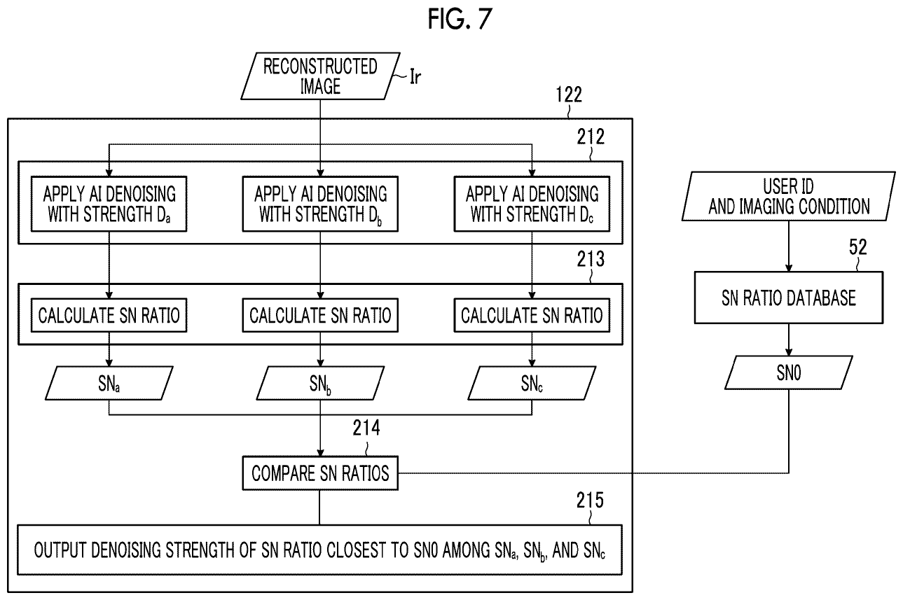

FIG. 7 is a functional block diagram illustrating a first configuration example of the denoising strength acquisition unit illustrated in FIG. 2. The denoising strength acquisition unit 122 comprises a denoised image generation unit 212, an SN ratio calculation unit 213, an SN ratio comparison unit 214, and a denoising strength output unit 215.

The denoised image generation unit 212 generates a plurality of provisional denoised images by applying a plurality of denoising strengths different from each other to a reconstructed image Ir generated by using the image reconstruction unit 102 illustrated in FIG. 2. FIG. 7 illustrates an example in which three types of a denoising strength Dₐ, a denoising strength D_{b}, and a denoising strength D_{c} different from each other are applied, and three types of provisional denoised images are generated.

The SN ratio calculation unit 213 calculates the SN ratio of each of the plurality of denoised images generated by using the denoised image generation unit 212. FIG. 7 illustrates an example in which SNₐ, SN_{b}, and SN_{c} are calculated as the SN ratios of the denoised images to which the denoising strength Dₐ, the denoising strength D_{b}, and the denoising strength D_{c} are applied, respectively.

The SN ratio comparison unit 214 compares the SN ratio SN0 acquired as the SN ratio preferred by the user and corresponding to the imaging condition with the SN ratio SNₐ, the SN ratio SN_{b}, and the SN ratio SN_{c} calculated by the SN ratio calculation unit 213. In a case in which any of the SN ratio SNₐ, the SN ratio SN_{b}, or the SN ratio SN_{c} is identical to the SN ratio SN0, the identical SN ratio to the SN ratio SN0 is the comparison result.

In a case in which the SN ratio SN0 matches none of the SN ratio SNₐ, and the SN ratio SN_{b}, and the SN ratio SN_{c}, the SN ratio closest to the SN ratio SN0 among the SN ratio SNₐ, the SN ratio SN_{b}, and the SN ratio SN_{c} is used as the comparison result. The SN ratio in which an absolute value of the difference from the SN ratio SN0 is the smallest is applied to the SN ratio closest to the SN ratio SN0.

The denoising strength output unit 215 outputs the denoising strength applied to the provisional denoised image in which the SN ratio, which is the comparison result of the SN ratio comparison unit 214, is calculated. For example, in a case in which the SN ratio SNₐ is identical to the SN ratio SN0 or closest to the SN ratio SN0, the denoising strength output unit 215 outputs the denoising strength Dₐ.

### Specific Example of SN Ratio Calculation Method

A specific example of the SN ratio calculation method applied to the SN ratio calculation unit 213 will be described. In the MR image, the SN ratio can be calculated using a characteristic that a noise level of a background is proportional to a noise level of the entire MR image.

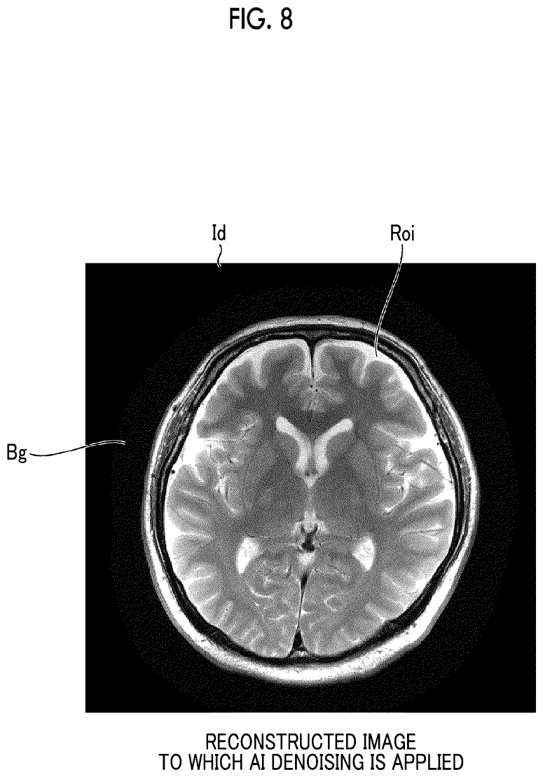

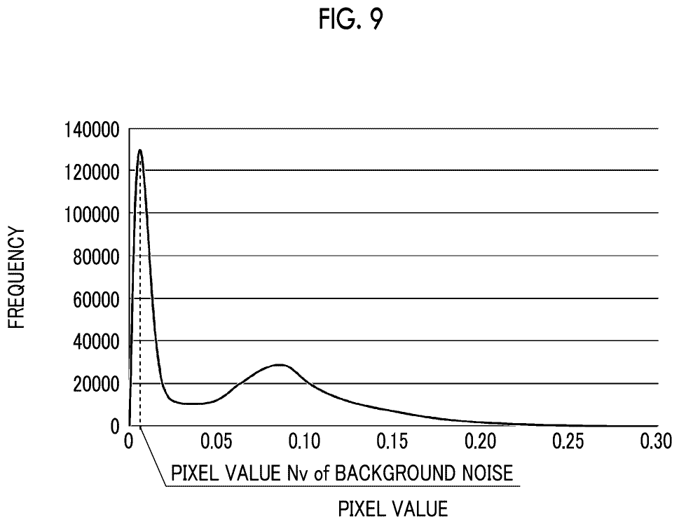

FIG. 8 is an explanatory diagram of a background of a region of interest. FIG. 8 illustrates a region of interest Roi and a background Bg in a denoised image Id, which is the reconstructed image to which the AI denoising is applied. FIG. 9 is an explanatory diagram of a pixel value of background noise. FIG. 9 illustrates a histogram of pixel values of the denoised image Id. In a graph illustrating the histogram of the pixel values of the denoised image Id, a horizontal axis represents a pixel value, and a vertical axis represents a frequency.

The SN ratio calculation unit 213 illustrated in FIG. 7 extracts the region of interest Roi of the denoised image Id illustrated in FIG. 8, extracts the background Bg of the region of interest Roi, and estimates the pixel value of the noise of the background Bg. The background Bg of the region of interest Roi is understood as an air region that covers a periphery of an organ or the like that is the region of interest Roi. A pixel value at which the frequency in the histogram of the pixel values of the denoised image Id is a peak is applied to a pixel value Nv of the noise of the background Bg illustrated in FIG. 9. It should be noted that the pixel value Nv of the noise is understood as a signal value of the noise.

The SN ratio calculation unit 213 calculates a median value of the pixel values exceeding 0 in the denoised image Id, as a signal value Sv in a case of calculating the SN ratio. The signal value Sv may be an average value of the pixel values exceeding 0 in the denoised image Id. The SN ratio calculation unit 213 calculates Sv/Nv as the SN ratio of the denoised image Id.

It should be noted that the plurality of provisional denoised images generated by using the denoised image generation unit 212 illustrated in FIG. 7 are examples of a third denoised image. The SN ratio SNₐ, the SN ratio SN_{b}, and the SN ratio SN_{c} illustrated in the same drawing are examples of a third signal-to-noise ratio, and the denoising strength Dₐ output from the denoising strength output unit 215 is an example of a third denoising strength.

### Adjustment of Denoising Strength in Standardization Processing

The denoising strength is adjusted in the standardization processing in the standardization unit 104 illustrated in FIG. 2 (JP2022-34774). An input image input to the standardization unit 104 is denoted by I₀, and an output image output from the standardization unit 104 is denoted by Iₙ.

In a case in which the median value of the pixel values exceeding 0 in the input image I₀ is denoted by Minput(I₀), and the pixel value of the background noise is denoted by Nv, the SN ratio SNR is represented by SNR=Minput(I₀)/Nv.

In a case in which the denoising strength is denoted by D, the output image Iₙ of the standardization unit 104 is represented by Iₙ={1/Minput(I₀)}×{a×(SNR/D)+b}×I₀. It should be noted that a and b are defined constants derived by performing experiments or the like.

For example, in a case in which the denoising strength is adjusted in three stages of weak, medium, and strong, the value of the denoising strength D that functions as a coefficient for determining the denoising strength in the output image Iₙ is set to 0.5, 1.0, and 1.5.

### Stageless Type

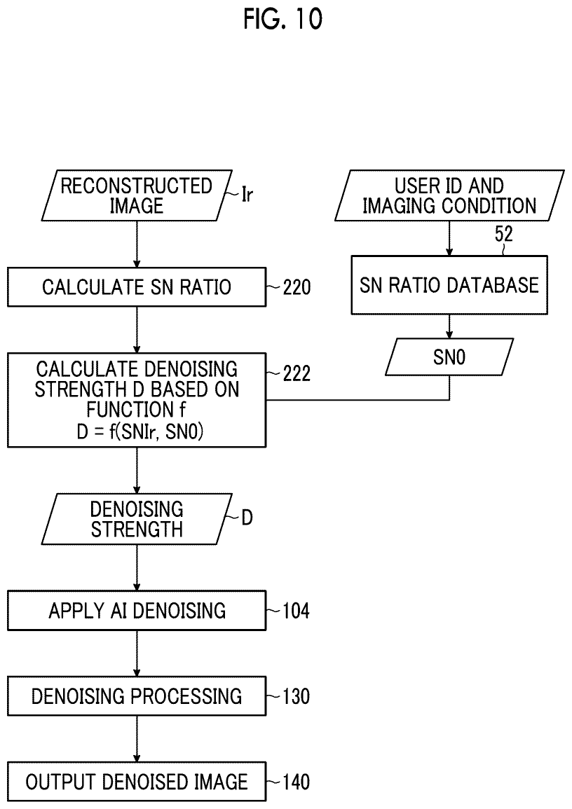

FIG. 10 is a functional block diagram illustrating a second configuration example of the denoising strength acquisition unit illustrated in FIG. 2. The denoising strength acquisition unit 122 illustrated in FIG. 2 comprises an SN ratio calculation unit 220 and a denoising strength calculation unit 222. The SN ratio calculation unit 220 calculates the SN ratio SNIr of the reconstructed image Ir generated by the image reconstruction unit 102 illustrated in FIG. 2.

The denoising strength calculation unit 222 calculates the denoising strength D represented by a function f(SNIr, SN0) in which the SN ratio SNIr of the reconstructed image Ir and the SN ratio SN0 preferred by the user are used as the parameters. The denoising strength D calculated by the denoising strength calculation unit 222 is applied to the standardization processing of the reconstructed image Ir performed by the standardization unit 104.

The reconstructed image Ir subjected to the standardization processing is subjected to the denoising processing by the denoising processing unit 130 using the learning model 132 illustrated in FIG. 2, and is output by using the denoised image output unit 140.

It should be noted that the SN ratio SNIr output by the SN ratio calculation unit 220 illustrated in FIG. 10 is an example of a fourth signal-to-noise ratio. In addition, the denoising strength D illustrated in the same drawing is an example of a fourth denoising strength.

### Specific Example of Calculation of Denoising Strength

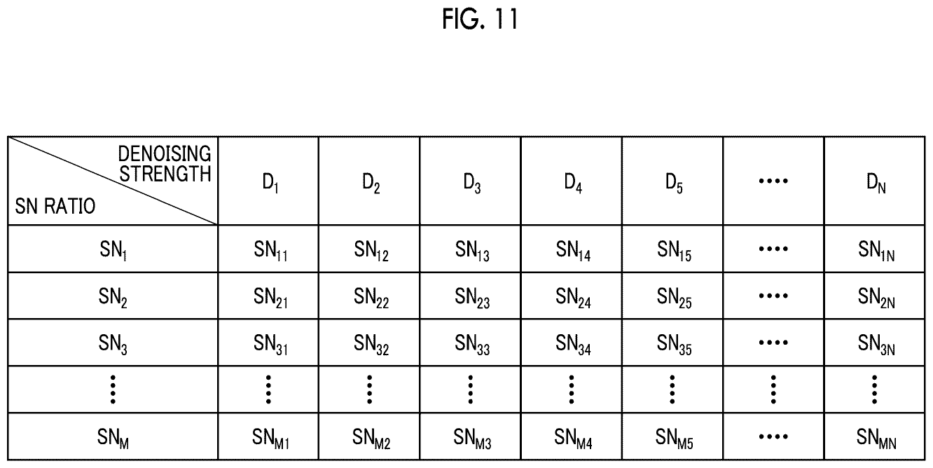

FIG. 11 is a table illustrating a result of calculating the SN ratio of the denoised image generated by applying the plurality of denoising strengths to a numerical phantom having different SN ratios. The table illustrated in FIG. 11 illustrates a case in which the number of SN ratios of the numerical phantom is M and the number of denoising strengths is N. M and N each represent an integer of 1 or more.

A value in a range of the SN ratio of the reconstructed image Ir, which is the target of the denoising processing, is applied to each of the SN ratio SN₁ to the SN ratio SN_{M} of the numerical phantom illustrated in FIG. 11. In addition, a value in a range used for the denoising processing on the reconstructed image Ir is applied to each of the denoising strength D₁ to the denoising strength D_{N}.

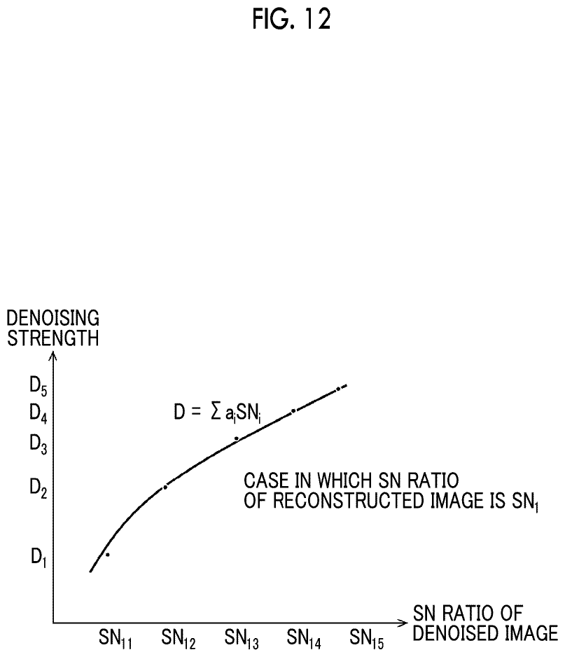

FIG. 12 is an explanatory diagram of a function representing the denoising strength. FIG. 12 illustrates a case in which the SN ratio of the numerical phantom is SN₁, and the denoising strength D=Σ(aᵢ×SNᵢ) is illustrated as a graph for a range of the denoising strength from Di to D₅. It should be noted that i is a subscript of 11 to MN, and aᵢ is a constant defined in advance.

A function representing the denoising strength D illustrated in FIG. 12 is stored in advance for each of the SN ratio SN₁ to the SN ratio SN_{M} illustrated in FIG. 11. The function representing the denoising strength D may be stored in a storage device provided in the computer 20, or may be stored in an external device, such as the external storage device 50. It should be noted that the function representing the denoising strength D illustrated in the same drawing is an example of a function representing a relationship between a signal-to-noise ratio and a denoising strength.

The denoising strength calculation unit 222 illustrated in FIG. 10 searches for the SN ratio that is identical to the SN ratio SNIr of the reconstructed image Ir calculated by the SN ratio calculation unit 220 or has the smallest absolute value of the difference from the SN ratio SNIr, from among the SN ratio SN₁ to the SN ratio SN_{M} illustrated in FIG. 11. The denoising strength calculation unit 222 determines the value of the denoising strength D, which is the SN ratio SN0 preferred by the user, by using the function for calculating the denoising strength D=Σ(aᵢ×SNᵢ) corresponding to the SN ratio obtained as the search result.

### First Modification Example of Embodiment

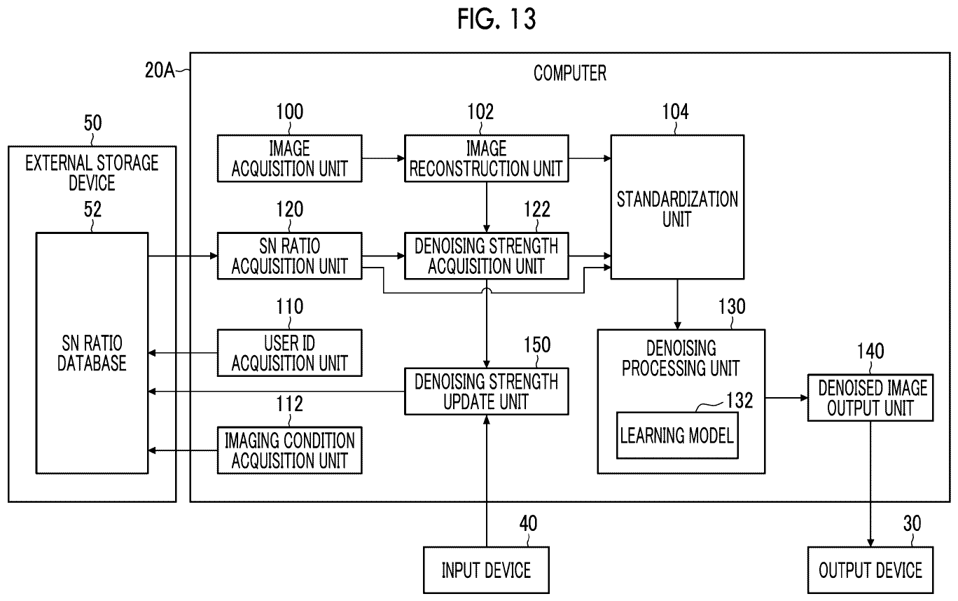

FIG. 13 is a functional block diagram illustrating an electric configuration of a computer according to a first modification example. A computer 20A illustrated in FIG. 13 has a function of updating the SN ratio database stored in the external storage device 50. That is, the computer 20A comprises a denoising strength update unit 150 that updates the SN ratio database.

The denoising strength update unit 150 updates the SN ratio preferred by the user and stored in the SN ratio database 52 in a case in which an input signal indicating the update of the denoising strength input by using the input device 40 is acquired.

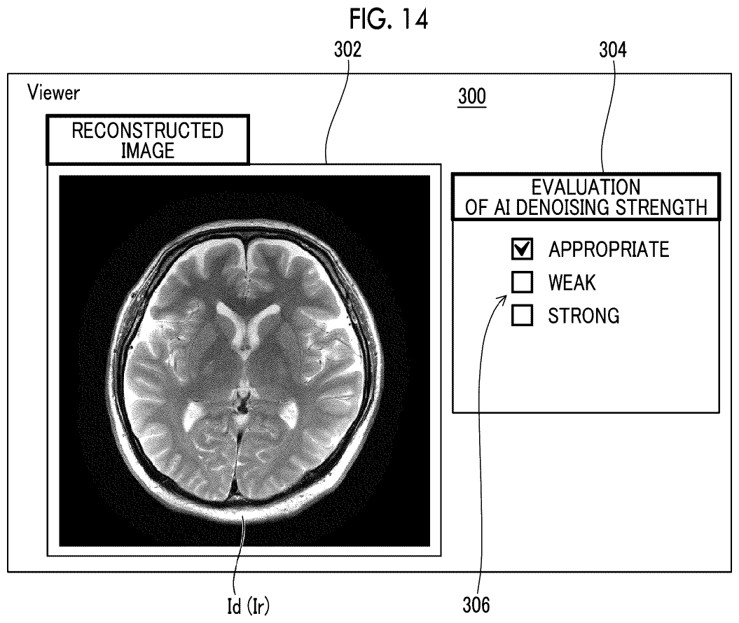

FIG. 14 is an explanatory diagram illustrating an example of a user interface applied to feedback of the denoising strength. A viewer screen 300 illustrated in FIG. 14 is displayed by using the display which is the output device 30 illustrated in FIG. 13.

The viewer screen 300 includes an image display region 302 in which the reconstructed image Ir, which is the denoised image Id obtained by the denoising processing, is displayed, and an evaluation display region 304 in which the evaluation of the denoising strength is displayed. In FIG. 14, as the evaluation of the denoising strength, appropriate, weak, and strong are displayed, and a check box 306 is displayed for each of the three types of evaluations.

The user who views the denoised image Id displayed on the viewer screen 300 can check any of a plurality of check boxes 306 displayed in the evaluation display region 304 by using the mouse constituting the input device 40. The check of the user is fed back to the next and subsequent imaging.

In a case in which the denoising strength is changed in accordance with the result of the feedback and the denoising strength after the change is selected as being appropriate, in the next and subsequent imaging, the SN ratio preferred by the user and stored in the SN ratio database 52 is automatically updated. That is, the SN ratio of the denoised image, which is evaluated as being appropriate for the denoising strength, is stored in the SN ratio database 52 as a new SN ratio preferred by the user.

It should be noted that the denoised image evaluated as being appropriate for the denoising strength described in the embodiment is an example of a second denoised image. The denoising strength applied to the second denoised image is an example of a second denoising strength. The output device 30 that displays the viewer screen 300 illustrated in FIG. 14 is an example of a display device, and the check boxes 306 illustrated in the same drawing is an example of options representing the evaluation of the first denoising strength. The signal representing the evaluation of the first denoising strength described in the embodiment is an example of a first evaluation signal.

### Second Modification Example of Embodiment

A computer according to a second modification example has a function of displaying a preview of the denoised image Id in a case of automatically adjusting the denoising strength. The imaging is started in a case in which there is no problem with the denoising strength of the denoised image Id as the preview. On the other hand, the user changes the denoising strength in a case in which there is a problem with the denoising strength. In a case in which the denoising strength is changed, the SN ratio preferred by the user and stored in the SN ratio database 52 is automatically updated.

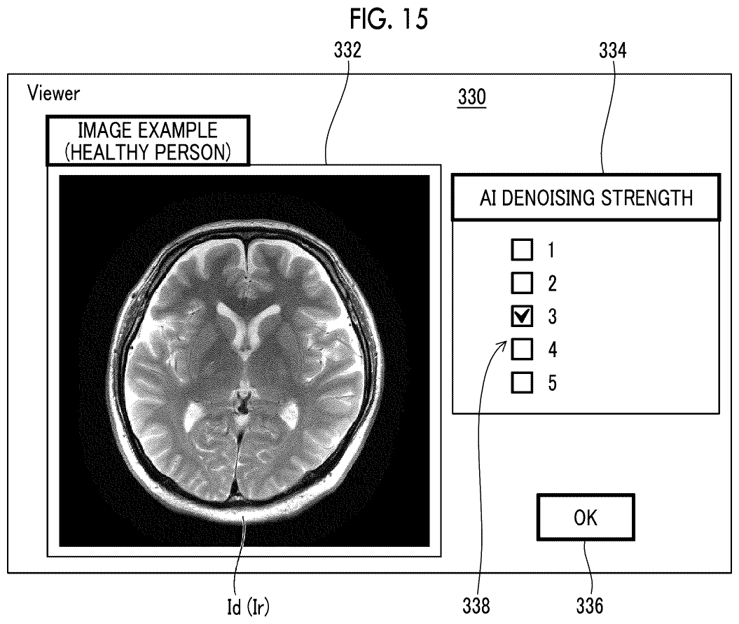

FIG. 15 is an explanatory diagram illustrating a first example of a confirmation screen before the automatic adjustment of the denoising strength. A confirmation screen 330 according to the first example includes an image display region 332 in which the preview of the denoised image Id is displayed, a denoising strength evaluation input region 334 in which the evaluation of the denoising strength is input, and an OK button 336 that is clicked in a case of deciding the input of the denoising strength evaluation input. The denoising strength evaluation input region 334 includes five check boxes 338 that are checked in a case in which any of the five levels of the denoising strength is selected.

The denoised image Id illustrated in FIG. 15 is a past MR image obtained by imaging any healthy person, and is a past MR image to which the imaging condition that is identical to or close to the imaging condition of the current imaging is applied to the MR image of the identical part to the current imaging target. The denoising processing in which the automatically adjusted denoising strength is applied is performed on the denoised image illustrated in the same drawing.

The user who views the preview of the denoised image Id displayed in the image display region 332 can change the denoising strength by checking any of the five check boxes 338 and further clicking the OK button 336. On the other hand, in a case in which there is no problem with the denoising strength of the preview of the denoised image Id, the user clicks the OK button 336 without changing the checked check box 338, and maintains the automatically set denoising strength to start the imaging.

It should be noted that the past MR image displayed on the confirmation screen 330 illustrated in FIG. 15 is an example of a fifth denoised image, and is an example of a normal image obtained by imaging a normal imaging target in the past. The imaging condition of the imaging of this time, which is identical or close to the imaging condition described in the embodiment, is an example of a fifth imaging condition that is identical to the first imaging condition or a fifth imaging condition that is close to the first imaging condition. The evaluation signal described in the embodiment in a case in which the user changes the check is an example of a fifth evaluation signal, and the denoising strength in a case in which the user changes the check is an example of a fifth denoising strength.

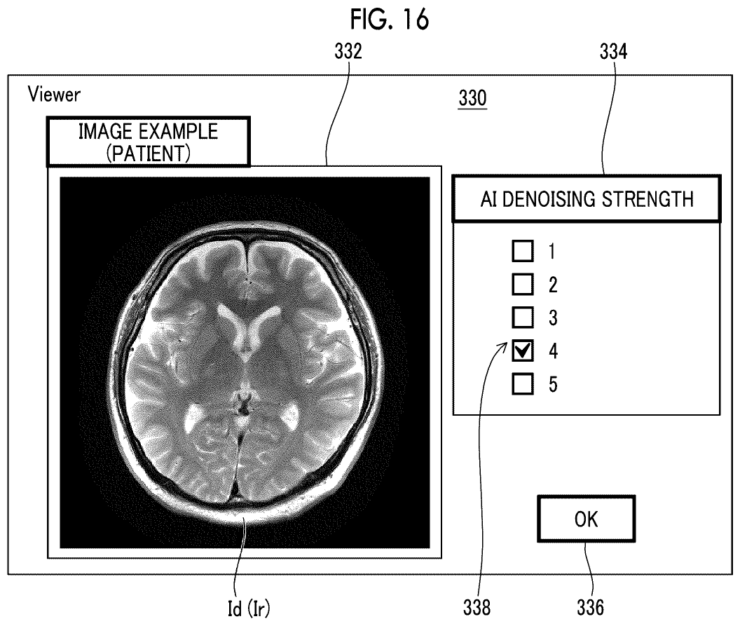

FIG. 16 is an explanatory diagram illustrating a second example of the confirmation screen before the automatic adjustment of the denoising strength. In the image display region 332 illustrated in FIG. 16, the denoised image Id obtained by performing the denoising processing in which the denoising strength automatically adjusted in accordance with the SN ratio preferred by the user is applied to the MR image that is identical to the imaging condition or close to the imaging condition among the MR images obtained by being captured in the past for the subject identical to the subject of the imaging target is displayed.

The denoising strength applied to the denoised image Id displayed in the image display region 332 is checked in the denoising strength evaluation input region 334 and displayed. The user can view the denoised image Id displayed in the image display region 332 to determine whether or not the denoising strength is appropriate.

In a case in which the user determines that the denoising strength is appropriate, and the user clicks the OK button 336, the automatically set denoising strength is maintained, and the imaging is started. On the other hand, in a case in which the user determines that the change of the denoising strength is necessary and the check box that is not checked is checked, the denoised image Id to which the checked denoising strength is applied is displayed again.

In a case in which it is determined that the denoising strength applied to the denoised image Id displayed again is appropriate, the imaging is started, and the SN ratio preferred by the user and stored in the SN ratio database 52 is automatically updated.

It should be noted that the subject identical to the subject of the imaging target described in the embodiment is an example of an identical imaging target. The MR image obtained by being captured in the past and illustrated in FIG. 16 is an example of an identical-imaging target image, and the denoised image obtained by performing the denoising processing on the MR image obtained by being captured in the past is an example of a sixth denoised image.

The denoising strength applied to the denoising processing of the MR image generated by being captured in the past illustrated in FIG. 16 is an example of a sixth denoising strength. The imaging condition applied to the MR image generated by being captured in the past and illustrated in the same drawing is an example of a sixth imaging condition that is identical to the first imaging condition or a sixth imaging condition that is close to the first imaging condition. The evaluation signal described in the embodiment in a case in which the user changes the check is an example of a sixth evaluation signal.

### Actions and Effects of Embodiment

The following actions and effects can be obtained with the MRI apparatus 1 according to the embodiment.

[1]

For each user such as the doctor, the SN ratio of the reconstructed image Ir generated by being captured in the past is referred to, the SN ratio SN0 preferred by the user is acquired, and the denoising strength D applied to the denoising processing on the reconstructed image Ir newly obtained by being captured is automatically set in accordance with the SN ratio SN0 preferred by the user.

As a result, the denoising processing in which the denoising strength D corresponding to the SN ratio SN0 preferred by the user and stored in advance is applied is executed, and the preferable denoised image Id is acquired.

[2]

In a case in which the denoising strength D is automatically set, the SN ratio SN0 preferred by the user is acquired, and the denoising strength D applied to the denoised image Id having the SN ratio that is identical to the SN ratio SN0 preferred by the user or the denoising strength D applied to the denoised image Id having the SN ratio close to the SN ratio SN0 preferred by the user is determined from the multi-stage denoising strength. As a result, the denoising processing in which the denoising strength D corresponding to the SN ratio SN0 preferred by the user among the plurality of denoising strengths D prepared in advance is applied is executed, and the preferable denoised image Id is acquired.

[3]

In a case in which the denoising strength D is automatically set, the SN ratio preferred by the user is acquired, and the denoising strength D corresponding to the SN ratio SN0 preferred by the user is determined by applying the function f (SNIr, SN0) representing the denoising strength in which the SN ratio is used as the parameter. As a result, the denoising processing in which the denoising strength D corresponding to the SN ratio preferred by the user among the denoising strengths D represented by the function f (SNir, SN0) in which the SN ratio is used as the parameter is applied is executed, and the preferable denoised image Id is acquired.

[4]

The preview of the denoised image Id is displayed by using the display that functions as the output device 30. The viewer screen 300 on which the preview is displayed has the check box 306 for inputting the evaluation of the denoising strength D of the denoised image Id.

As a result, the user who views the preview of the denoised image Id can input the evaluation of the denoising strength D of the denoised image Id by clicking the check box 306.

[5]

In a case in which the evaluation indicating that the denoising strength is appropriate is input for the denoised image Id in which the denoising strength D is changed, the SN ratio preferred by the user and stored in the SN ratio database 52 is updated. As a result, the SN ratio preferred by the user and stored in the SN ratio database 52 is updated in accordance with the evaluation of the user with respect to the denoising strength.

[6]

The denoised image Id subjected to the denoising processing in which the denoising strength D automatically set for the past image of the identical part obtained by imaging the healthy person in the past is applied is displayed along with the denoising strength D on the confirmation screen 330 as the setting screen of the imaging condition before the imaging. The imaging is started in a case in which there is no problem with the denoising strength D. On the other hand, the denoising strength D is changed in a case in which there is a problem with the denoising strength D. In a case in which the denoising strength D is changed, the SN ratio preferred by the user and stored in the SN ratio database 52 is updated.

As a result, the denoising strength can be confirmed in advance. In addition, the SN ratio preferred by the user and stored in the SN ratio database 52 is updated in response to the change of the denoising strength.

[7]

The denoised image Id of the past image to which the imaging condition identical to the current imaging condition is applied or the past image to which the imaging condition close to the current imaging condition is applied among the past images of the subject as the imaging target is displayed along with the denoising strength D on the confirmation screen 330 as the setting screen of the imaging condition before the imaging.

As a result, the denoising strength applied to the denoising processing of the past image can be confirmed, and the user can determine the appropriateness of the denoising strength D applied to the denoising processing for the current imaging.

[8]

The confirmation screen 330 displays the check box for each of the plurality of denoising strengths D. As a result, the user can input a command to change the denoising strength D by checking the check box using the input device 40.

[9]

In a case in which the denoising strength D is changed, the SN ratio preferred by the user and stored in the SN ratio database 52 is updated. As a result, the SN ratio preferred by the user and stored in the SN ratio database 52 is updated in accordance with the change of the denoising strength.

### Hardware Configuration of each Processing Unit

The hardware structure of the processing unit that executes various types of processing, such as the image acquisition unit 100, the image reconstruction unit 102, the standardization unit 104, the user ID acquisition unit 110, the imaging condition acquisition unit 112, the SN ratio acquisition unit 120, the denoising strength acquisition unit 122, the denoising processing unit 130, and the denoised image output unit 140 in the computer 20 illustrated in FIG. 2 and the computer 20A illustrated in FIG. 13 is, for example, following various processors.

The various processors include, for example, a CPU which is a general-purpose processor executing a program to function as various processing units, a GPU, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform specific processing.

One processing unit may be configured by one of these various processors or by two or more processors of the same type or different types. For example, one processing unit may be configured by a plurality of FPGAs, a combination of a CPU and an FPGA, or a combination of a CPU and a GPU. Moreover, a plurality of processing units may be configured by one processor. A first example of the configuration in which a plurality of processing units are configured by one processor is a form in which one processor is configured by a combination of one or more CPUs and software and functions as a plurality of processing units, as represented by a client computer or a server computer. A second example of the configuration is a form in which a processor that implements the functions of the entire system including a plurality of processing units using one integrated circuit (IC) chip is used, as represented by a system on chip (SoC). As described above, various processing units are configured by using one or more of the various processors as the hardware structure.

Further, the hardware structure of these various processors is an electric circuit (circuitry) obtained by combining circuit elements such as semiconductor elements.

### Program for Operating Computer

A program for causing a computer to realize some or all of the processing functions of each of the computer 20 according to the embodiment and the computer 20A according to the modification example of the embodiment can be recorded in a computer-readable medium, such as an optical disk, a magnetic disk, a semiconductor memory, or another non-transitory information storage medium that is a tangible object, and the program can be provided via the information storage medium.

In addition, instead of the aspect of providing the program by storing the program in the non-transitory computer-readable medium that is the tangible object, it is also possible to provide the program as a program signal by using an electric communication line such as the Internet.

Further, some or all of the processing functions in the computer 20 or the like may be realized by cloud computing, or may be provided as software as a service (SaaS).

The present disclosure is not limited to the above-described embodiment, and various modifications can be made without departing from the technical idea of the present disclosure.

### EXPLANATION OF REFERENCES

20: computer
30: output device
50: external storage device
52: SN ratio database
100: image acquisition unit
102: image reconstruction unit
104: standardization unit
110: user ID acquisition unit
112: imaging condition acquisition unit
120: SN ratio acquisition unit
122: denoising strength acquisition unit
130: denoising processing unit
132: learning model
140: denoised image output unit

What is claimed is:

1. An image processing apparatus comprising:
a processor configured to:
acquire user identification information;
acquire a first imaging condition applied to an imaging apparatus;
acquire a first image generated by being captured using the imaging apparatus to which the first imaging condition is applied;
set a first denoising strength applied to denoising processing on the first image; and
execute the denoising processing on the first image by applying the first denoising strength, to generate a first denoised image,
wherein the processor is configured to:
acquire, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition, as a first signal-to-noise ratio; and
set a denoising strength derived based on the first signal-to-noise ratio, as the first denoising strength.

2. The image processing apparatus according to claim 1,
wherein the processor is configured to acquire the first signal-to-noise ratio corresponding to the second imaging condition that is identical to the first imaging condition or the second imaging condition that is close to the first imaging condition, with reference to a signal-to-noise ratio database in which the plurality of second imaging conditions are stored in association with the plurality of second signal-to-noise ratios.

3. The image processing apparatus according to claim 2,
wherein the processor is configured to, in a case in which the first denoising strength is changed, update the signal-to-noise ratio database in accordance with a second denoising strength after the change.

4. The image processing apparatus according to claim 1,
wherein the processor is configured to:
execute a plurality of times of the denoising processing with different denoising strengths on the first image, to generate a plurality of third denoised images;
derive a third signal-to-noise ratio from each of the plurality of third denoised images; and
set a third denoising strength applied to the third denoised image in which the third signal-to-noise ratio having a smallest difference from the first signal-to-noise ratio is derived, as the first denoising strength.

5. The image processing apparatus according to claim 1,
wherein the processor is configured to:
calculate a fourth signal-to-noise ratio from the first image; and
set a fourth denoising strength corresponding to the fourth signal-to-noise ratio, as the first denoising strength, by using a function representing a relationship between a signal-to-noise ratio and a denoising strength.

6. The image processing apparatus according to claim 1,
wherein the processor is configured to:
display, on a display device, the first denoised image;
display, on the display device, options representing an evaluation of the first denoising strength applied to the denoising processing on the first denoised image;
acquire a first evaluation signal representing the evaluation of the first denoising strength selected from among the options; and
perform feedback of the evaluation of the first denoising strength represented by the first evaluation signal with respect to the setting of the first denoising strength.

7. The image processing apparatus according to claim 1,
wherein the processor is configured to:
display, on a display device, a fifth denoised image generated by executing the denoising processing by applying the first denoising strength to a past image generated by being captured in the past by applying a fifth imaging condition that is identical to the first imaging condition or a fifth imaging condition that is close to the first imaging condition;
acquire a fifth evaluation signal representing an evaluation of a fifth denoising strength applied to the denoising processing on the fifth denoised image; and
determine whether to maintain or change the first denoising strength in accordance with the evaluation of the fifth denoising strength represented by the fifth evaluation signal.

8. The image processing apparatus according to claim 7,
wherein a normal image generated by imaging a normal imaging target in the past is applied to the past image.

9. The image processing apparatus according to claim 7,
wherein an identical-imaging target image generated by imaging an identical imaging target in the past by applying a sixth imaging condition that is identical to the first imaging condition or a sixth imaging condition that is close to the first imaging condition is applied to the past image, and
the processor is configured to display, on the display device, a sixth denoised image obtained by executing the denoising processing on the identical-imaging target image, and a sixth denoising strength applied to the denoising processing executed on the identical-imaging target image.

10. The image processing apparatus according to claim 9,
wherein the processor is configured to:
acquire a sixth evaluation signal representing an evaluation of the sixth denoising strength; and
determine whether to maintain or change the first denoising strength in accordance with the evaluation of the sixth denoising strength represented by the sixth evaluation signal.

11. An operation method of an image processing apparatus to which a computer provided with a processor is applied, the operation method comprising:
via the processor,
acquiring user identification information;
acquiring a first imaging condition applied to an imaging apparatus;
acquiring a first image generated by being captured using the imaging apparatus to which the first imaging condition is applied;
setting a first denoising strength applied to denoising processing on the first image; and
executing the denoising processing on the first image by applying the first denoising strength, to generate a first denoised image,
wherein, in a case of setting the first denoising strength,
from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition is acquired as a first signal-to-noise ratio, and
a denoising strength derived based on the first signal-to-noise ratio is set as the first denoising strength.

12. A non-transitory, computer-readable tangible recording medium which records thereon a program for causing, when read by a computer, the computer to realize:
a function of acquiring user identification information;
a function of acquiring a first imaging condition applied to an imaging apparatus;
a function of acquiring a first image generated by being captured using the imaging apparatus to which the first imaging condition is applied;
a function of setting a first denoising strength applied to denoising processing on the first image; and
a function of executing the denoising processing on the first image by applying the first denoising strength, to generate a first denoised image,
wherein, in the function of setting the first denoising strength,
from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition is acquired as a first signal-to-noise ratio, and
a denoising strength derived based on the first signal-to-noise ratio is set as the first denoising strength.

13. A medical image processing system comprising:
an image processing apparatus configured to execute defined image processing on a medical image generated by imaging a subject using an imaging apparatus,
wherein the image processing apparatus includes a processor configured to:
acquire user identification information;
acquire a first imaging condition applied to an imaging apparatus;
acquire a first image generated by being captured using the imaging apparatus to which the first imaging condition is applied;
set a first denoising strength applied to denoising processing on the first image; and
execute the denoising processing on the first image by applying the first denoising strength, to generate a first denoised image, and
the processor is configured to:
acquire, from among a plurality of second signal-to-noise ratios derived from a plurality of second images generated by a plurality of times of past imaging for each user to which a plurality of second imaging conditions are applied, a second signal-to-noise ratio corresponding to a second imaging condition that is identical to the first imaging condition or a second imaging condition that is close to the first imaging condition, as a first signal-to-noise ratio; and
set a denoising strength derived based on the first signal-to-noise ratio, as the first denoising strength.
